(19) United States
(12) Patent Application Publication
(10) Pub. No.: US 2005/0247312 A1
(43) Pub. Date: Nov. 10, 2005

(54) Medicament dispenser

(76) Inventor: Michael Birsha Davies, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/522,324

(22) PCT Filed: Jul. 23, 2003
(86) PCT No.: PCT/EP03/08150

§ 371 Date: Jan. 25, 2005

(30) Foreign Application Priority Data
Jul. 25, 2002 (GB) 0217199.9

(51) Int. Cl. B05D 7/14; A61M 15/00; B65D 83/06; A61M 16/00

(52) U.S. Cl. 128/203.15

(57) ABSTRACT

A medicament dispenser device for use in the delivery of a combination medicament product is disclosed. The device includes a first medicament container for containing a first medicament component; a first release means for releasing the contents of the first medicament container; at least one further medicament container for containing at least one further medicament component; and at least one further release means for releasing the contents of each said at least one further medicament container. The first medicament component is kept separate from the at least one further medicament component until the point of release thereof for delivery in combination. The dispenser device can additionally include at least one actuation indicator associated with the first medicament container and the at least one further medicament container.

### TECHNICAL FIELD

The present invention relates to a medicament dispenser for dispensing medicament combination products. The invention particularly relates to a device for use in monitoring and counting the dispensing of combination medicament products.

### BACKGROUND TO THE INVENTION

The use of inhalation devices in the administration of medicaments, for example in bronchodilation therapy is well known. Such devices generally comprise a body or housing within which a medicament carrier is located. Known inhalation devices include those in which the medicament carrier is a blister strip containing a number of discrete doses of powdered medicament. Such devices usually contain a mechanism of accessing these doses, usually comprising either piercing means or means to peel a lid sheet away from a base sheet. The powdered medicament can then be accessed and inhaled. Other known devices include those in which the medicament is delivered in aerosol form, including the well known metered dose inhaler (MDI) delivery devices. Liquid-based inhaler devices are also known.

Therapies involving combinations of different and complementary active medicaments are known. These can be administered either as distinct combination (i.e. multi-active) medicament products, which comprise a defined mixture of each component medicament, or as groups of single active medicament products, which are designed to be taken in combination or sequentially. Whilst combination products offer added convenience for the patient, certain medicament actives are difficult to formulate as distinct combination products. For example, the actives may interact chemically with each other in an undesirable way when formulated together.

It is thus, desirable in certain circumstances, to have a medicament dispenser that separately (i.e. in isolated fashion) contains each active component of a combination product, but which enables the delivery of a combined dose in response to a minimum number of patient actions. In particular, it is desirable that each active component of the combined dose is delivered to the patient in a single, combined dose in response to a single patient dosing action. For example, it is desirable that a combined product for inhalation be delivered in response to a single patient actuation of an inhaler, even where the active components of that combined product are separately stored within the inhaler device.

The Applicants have also observed that particular medicaments can be more suited to delivery to by particular types of inhaler device. For example, one particular medicament may be more suitable for delivery by an MDI device, whereas another may be more suitable for delivery by a DPI device. That suitability may for example, be driven by ease of formulation of the medicament for that particular inhaler device or by the delivery and pharmaceutical performance characteristics obtainable when the particular inhaler device is employed. Unitary devices comprising different types of dispenser are thus, envisaged.

It is highly desirable that any particular multi-dose delivery device is configured to provide the patient with feedback relating either to how many doses of medicament have been delivered from the device or often more importantly, how many doses remain within the device. Thus, various dose count systems have been developed for use with different types of medicament delivery device. Both mechanical and electronic counters are known and also both analogue and digital count displays.

The Applicants have now realised that dose counting presents particular problems when a combination medicament delivery device configured to accommodate separately located active components is employed. In one aspect, it is desirable to separately detect and count the actuation/dispensing of each active component from its separate location within the device. In another aspect, it is desirable to detect and count the actuation/dispensing of the muli-active component ‘combined product’ and therefore to present a single ‘combined dose’ count to the patient. In this latter case, which has the added advantage of ‘one count’ simplicity for the patient, it is important to ensure that a count is recorded only when all active components of the combined product are dispensed.

Having identified and appreciated the above problems, the Applicants have now devised solutions to them. Where a single ‘combined dose’ count is desired, coupling of various aspects of device actuation, dose counting and/or dose release sensing systems is provided. Where separate counting (i.e. for each individual component of the combination) is desired, improvements are presented which readily enable a patient to make checks on ‘combined dose’ counting. Suitably, the delivery of the combined medicament dose and counting thereof occurs on an essentially simultaneous basis and is responsive to a minimum number of patient actions (e.g. single patient actuation or inhalation step).

### SUMMARY OF THE INVENTION

According to one aspect of the invention there is provided a medicament dispenser device for use in the delivery of a combination medicament product, the device comprising
a first medicament container for containing a first medicament component;
a first release means for releasing the contents of said first medicament container;
at least one further medicament container for containing at least one further medicament component; and
at least one further release means for releasing the contents of each said at least one further medicament container;
wherein the first medicament component is kept separate from the at least one further medicament component until the point of release thereof for delivery in combination, and wherein the dispenser device additionally comprises
at least one actuation indicator associated with the first medicament container and/or the at least one further medicament container.

Suitably, the contents of the first and at least one further medicament container are released as a combination product (i.e. combining the first medicament component and the at least one further medicament component) for delivery to the patient.

Suitably, in combination, the first medicament and at least one further medicament comprise a defined combination product. That is to say, that when combined together the distinct active medicament doses released by actuation of the device form a dose of a ‘multi-active’ medicament treatment.

Suitably, the first medicament component and the at least one further medicament component are non-identical medicaments. In aspects, the first medicament container and at least one further medicament container are arranged (e.g. sized, shaped, designed) to contain the respective non-identical medicament components.

In aspects, each separate medicament component may be arranged for simultaneous or sequential release from the one or more medicament containers, although in general where components are released sequentially the time delay between release of each separate medicament component is short (e.g. milliseconds) to ensure that a combination product is provided for delivery to the patient.

On actuation, the dispenser device is designed to deliver a dose portion of the first medicament and a dose portion of each at least one further medicament. The term ‘dose portion’ is employed because in the context of the invention the distinct ‘portions’ are brought together on delivery to form a combination (i.e. multi-active) product dose.

In one particular aspect, the first medicament container contains plural co-formulation compatible medicament components, and each at least one further medicament container contains at least one co-formulation incompatible medicament component.

The term ‘co-formulation compatible’ herein is used to mean compatible in the sense of being amenable to co-formulation, perhaps even displaying synergetic co-formulation characteristics. The term ‘co-formulation incompatible’ is used to mean the reverse, that is to say for whatever reason including chemical or physical incompatibility or simply lack of synergetic characteristics or benefits, the medicament components are either non-amenable to co-formulation or for whatever reason, including for development simplicity, preferably not co-formulated.

In one particular aspect, the unitary device is designed to receive the first and only one further medicament dispenser (i.e. two medicament containers only). Thus, the device functions as a bi-dispenser device.

The medicament dispenser device is provided with at least one actuation indicator associated with the first medicament container and the at least one further medicament container. The association may be direct, or it may indirect, such as through some form of intermediary component such as a coupling component (e.g. mechanical or electronic) or indeed another medicament container.

The term ‘actuation indicator’ is used herein to mean any means for indicating, or in particular counting, when the dispenser device is actuated. The term ‘actuation’ is used to mean actuation of the dispenser device such that either medicament is delivered therefrom (e.g. by a firing step) or that of preliminary actuation of the dispenser device, which readies it for delivery (e.g. a priming step) such as one in which medicament is accessed to make it available for delivery and/or advanced to a delivery position within the dispenser device.

Actuation indication may thus in aspects, be based on detection of any actuation step, which will result in delivery of medicament from the dispenser device; or detection of medicament released by any actuation step; or detection of any movement which accesses or advances medicament dose to make it available for delivery.

The actuation indicator particularly includes means for registering and displaying dose release or dose count information to the patient. At a basic level, that information may simply relate to the fact that an actuation step or medicament release has been detected, but more often the information relates to the number of doses delivered or remaining of each medicament in the dispenser device. The information may be delayed in digital or analogue form, typically using standard count indicia (e.g. ‘999’ to ‘000’ indicia count display). Embodiments involving either ‘counting up’ or ‘counting down’ in increments are envisaged.

Dose release or dose count information may be displayed for the ‘combined product’ (i.e. first and at least one further medicament) together, or it may be separately displayed for each separate medicament component of the combination.

In one aspect, a single actuation indicator is associated with both the first medicament container and the at least one further medicament container. In another aspect, each medicament container has an actuation indicator associated therewith. In a further aspect, one medicament container has an actuation indicator associated therewith and a relevant movement of that container is coupled to that of at least one further container. Intermediate variations are also envisaged. Intermediate variations are also envisaged. As above, the association may be direct, or it may be through some form of intermediary component such as one or more coupling components.

In a first particular aspect, a single actuation indicator is provided, which is associated (e.g. directly) with the first medicament container. The first medicament container and at least one further medicament container are then coupled such any actuation of the first medicament container also results in actuation of the at least one further medicament container. The actuation indicator therefore detects and optionally, displays information relating to the actuation of all the so coupled medicament containers.

In one variation of the first particular aspect described above, the dispenser device is provided with a coupling actuator (e.g. an actuation mechanism) which acts such as to couple the actuation of the first and the at least one further medicament container. In one aspect, that coupling actuator comprises a button or lever which acts on both the first and the at least one further medicament container to (essentially simultaneously) release medicament therefrom. In this variation, it will be appreciated that the single actuation indicator may be conveniently positioned to detect the actuation of any of the medicament containers since when one is actuated, the other(s) are too.

In another variation of the first particular aspect described above, the first and the at least one further medicament container are coupled together such that an actuating movement of the first medicament container also results in an actuating movement of the at least one further medicament container. This variation is particularly relevant to metered dose inhaler (MDI) type devices in which, generally actuation is responsive to an actuating movement (e.g. push down the MDI canister) relative to its housing. In one aspect, the first and the at least one further medicament container are fixedly coupled together (e.g. strapped together or otherwise mounted to each other, possibly via a coupling element). This coupling enables essentially simultaneous actuation of both the first and at least one further medicament container. In this variation also, it will be appreciated that the single actuation indicator may be conveniently positioned to detect the actuation of any of the medicament containers since when one is actuated, the other(s) are too. Suitably, in this aspect actuation of the actuation indicator is responsive to the coupled actuating movement relevant to the first and at least one further medicament container.

In another variation of the first particular aspect described above the first and the at least one further medicament container are coupled together such that a metering movement relevant to the first medicament container also results in a metering movement relevant to the at least one further medicament container. This variation is particularly relevant to inhaler devices having a bulk reservoir from which doses are metered (in particular, reservoir dry powder inhalers (RDPI) and reservoir liquid spray inhalers) in which, generally metering is responsive to an metering movement (e.g. bring metering cavity into communication with the bulk reservoir) relative to the bulk reservoir. Suitably, in this aspect actuation of the actuation indicator is responsive to the coupled metering movement relevant to the first and at least one further medicament container.

In another variation of the first particular aspect described above the first and the at least one further medicament container are coupled together such that a dose advancement movement relevant to the first medicament container also results in a dose advancement movement relevant to the at least one further medicament container. This variation is particularly relevant to inhaler devices having multiple individual doses spaced on a carrier such as a blister pack (in particular, multi-dose dry powder inhalers (MDPI) as described hereinafter) in which, generally dose advancement to a delivery position is responsive to a dose advancement movement (e.g. advancing an elongate blister strip to move the next blistered dose to the delivery position) relative to a housing. Suitably, in this aspect actuation of the actuation indicator is responsive to the coupled dose advancement movement relevant to the first and at least one further medicament container.

In a further variation of the first particular aspect described above the dispenser device comprises a dose access coupling that acts such as to couple a dose access movement relevant to (e.g. movement relative to) the first and the at least one further medicament container. This variation is also particularly relevant to inhaler devices having multiple individual doses spaced on a carrier such as a blister pack (in particular, multi-dose dry powder inhalers (MDPI) as described hereinafter) in which the dose must first be accessed (e.g. by piercing or peeling of the pack) in order to make it available for delivery. Suitably, the first and the at least one further medicament container each comprise a pack carrying multiple individually accessible doses of medicament and said dose access movement enables access to a next accessible dose of each pack.

In one variation of the first particular aspect described above, the single actuation indicator is arranged to detect an actuation step relating to one (e.g. the first) medicament container. In another variation of the first particular aspect described above, the single actuation indicator is arranged to detect a pre-actuation step (e.g. dose access, dose advancement) relating to one (e.g. the first) medicament container. In a further variation, the single actuation indicator is arranged to detect the release of medicament from one (e.g. the first) medicament container.

In a second particular aspect, each medicament container is associated with its own actuation indicator. Each actuation indicator is therefore arranged to detect and optionally display information relating to the actuation of each medicament container. Any display may provide data relating to each individual medicament container, or more typically relevant to the delivery of a combined product.

In one variation of the second particular aspect described above, each actuation indicator is arranged to detect an actuation step relating to each medicament container. In another variation of the second particular aspect described above, each actuation indicator is arranged to detect a pre-actuation step (e.g. dose access, dose advancement) relating to each medicament container. In a further variation, each actuation indicator is arranged to detect the release of medicament from each medicament container.

When an actuation or pre-actuation (step) is to be detected, the dispenser device suitably may comprise an actuation sensor. The actuation sensor is for example, sensitive to parameters selected from the group consisting of electro magnetic radiation, magnetic field, light, motion, temperature, pressure, sound, oxygen concentration, carbon dioxide concentration and moisture. The actuation sensor is arranged to sense the actuation or pre-actuation of the dispenser. In one aspect, the actuation sensor is integral with the housing, for example moulded into a housing of the dispenser device or attached thereto. Alternatively, the actuation sensor is reversible attachable to the housing.

Where release of medicament is to be detected, the actuation indicator suitably comprises a release sensor for directly detecting the medicament release. The positioning of the release sensor in the dispenser device will be arranged to maximise detection of each, whilst minimising any interference effects (including those due to release of other medicament) and whilst minimising any effect on the delivery of each medicament to the patient.

Suitably, any sensor comprises an emitter and a detector. Altematively, any sensor comprises only a detector, for example a pyroelectric detector which responds to a decrease in temperature.

Suitably, the emitter emits electro magnetic radiation and the detector detects the electromagnetic radiation.

The electromagnetic radiation emitted from the emitter may be infrared, visible or ultraviolet radiation. Suitably, radiation in the range 0.95 μm to 0.35 μm is used. More suitably, the radiation is in the infrared range. In particular, infrared radiation with a wavelength of 0.88 μm has been found to be useful.

Suitably, the emitter is selected from the group consisting of light emitting diode, laser, incandescent lamp, electroluminescent and fluorescent light sources. Suitably, the emitter emits infra red radiation. In one embodiment, the emitter may include a filter, suitably an optical filter and preferably a polarising filter (particularly if the emitter is an incandescent source) in order to select a particular wavelength, a narrow range or ranges of wavelength. Several advantages may be obtained by selecting a particular wavelength or range/ranges of wavelength, for example a given range of wavelengths may be especially sensitive to a particular drug/propellant combination. Alternatively, one ‘sensitive’ range and one ‘insensitive’ range may be selected—in this case the ratio of the two or more wavelengths reaching the detector would be used to detect the drug, thus making the sensor less prone to errors caused by an overall reduction in intensity due to contamination of the optical path.

Suitably, the detector is selected from the group consisting of photodiode, phototransistor, light-dependent resistor and bolometer. Preferably, the detector detects infra red radiation. In one embodiment, the detector additionally comprises a filter, suitably an optical filter and preferably a polarising filter. The use of a filter will enable the wavelength/wavelengths detectable by the detector to be pre-determined giving advantages similar to those described for using a filter with the emitter, for example, the detector could be made sensitive only to the wavelengths chosen for the emitter so the detector could be less sensitive to extraneous light sources, such as room light/sun light. In a further embodiment, the detector is associated with an amplifier, since the output from the detector can be very small (of the order of micro Amps). Suitably, the amplifier is positioned as closed to the detector as possible to avoid amplifying any extraneous noise e.g. any electrical noise picked up in the connecting wires. In one particular embodiment, the amplifier is integrated with the detector, for example the detector and amplifier are positioned on the same integrated circuit or “chip”.

The detector may detect either an increase or decrease in radiation, compared to the amount of radiation emitted by the emitter. The increase or decrease may be due to interference of radiation reaching the detector by the medicament release.

In one aspect, the interference is due to absorption of radiation by the medicament release. In another aspect, the interference is due to scattering of radiation by the medicament release. In a further aspect, the interference is due to reflection of radiation by the medicament release. In a yet further aspect, the interference is due to refraction of radiation by the medicament release. In a still further aspect, the interference is due to diffraction of radiation by the medicament release.

In one aspect, the interference results in a decrease in the amount of radiation reaching the detector, for example due to absorption, scattering, refraction or diffraction, resulting in a decrease in the output signal. Alternatively, the amount of radiation reaching the detector may be maintained at a constant level by increasing the input level to the emitter. For example, an electronic feedback circuit that increases the current flowing through the emitter in order to maintain a constant flux at the detector may be used, resulting in an increase in the current supplied to the emitter as the medicament is released.

In a second aspect, the interference results in an increase in the amount of radiation reaching the detector, for example due to reflection by the medicament release, resulting in an increase in the output signal. Alternatively, the amount of radiation reaching the detector may be maintained at a constant level by decreasing the input level to the emitter. For example, an electronic feedback circuit that decreases the current flowing through the emitter in order to maintain a constant flux at the detector may be used, resulting in a decrease in the current supplied to the emitter as the medicament is released.

In one aspect, the emitter emits radiation of more than one wavelength and the detector detects radiation of more than one wavelength.

Suitably, any sensor can quantify the concentration of medicament within the medicament release by measuring radiation at more than one wavelength. These data can be processed, for example by a microprocessor, and compared against standardised data for a specified medicament to determine the concentration in the emission. For example, a first wavelength is used as a control to calibrate the system response. Suitably, this wavelength is not affected by the medicament release. A second wavelength is affected by the medicament release, for example due to interference of the radiation by the medicament release. The ratio of the amount of radiation of the first wavelength to the amount of radiation of the second wavelength arriving at the detector will depend on the concentration of medicament in the medicament release.

In one aspect, each release sensor is associated with a second release sensor (suitably having an emitter and a detector) for detecting a medicament release. Suitably, the second release sensor is positioned such that the medicament release passes each second sensor subsequent to passing each first release sensor. The presence of a second sensor may be used to increase confidence in the detection of the medicament release, for example for a detection to be considered valid, both sensors must be triggered. For example, a single release sensor may be “triggered” by a foreign body interrupting the radiation path, but in this case the second sensor would not be “triggered”; thus the detection would not be considered valid and a dose not shown as given. Furthermore, the time lapse between triggering of the first release sensor and triggering of the second release sensor may be used to determine whether a detection is valid, i.e. the second release sensor must be triggered within a specified time of the triggering of the first release sensor.

In one aspect, any sensor is integral with the outlet, for example moulded into a dispensing outlet of the dispenser device or attached thereto. In a second aspect, any sensor is reversibly attachable to the outlet and may be transferred from one outlet to another.

The actuation indicator may be associated mechanically or electronically with the actuation or release sensor(s), such that when the detector detects actuation or medicament release a signal is sent to the actuation indicator to record that a (part) dose has been dispensed.

In one aspect, the actuation indicator comprises a microprocessor. Suitably, the microprocessor performs operations on the data from any sensor and produces a signal output relating to the data or the outcome of an operation on the data.

Suitably, the actuation indicator additionally comprises a visual display unit for display of the data. Preferably, the visual display unit displays the number of doses of medicament used or remaining within the container. Preferably the doses are displayed numerically, by a series of coloured lights or by a monochrome bargraph.

The first and at least one further medicament containers may be of a similar-type or in aspects, be of a different type. This enables additional flexibility in that one container may for example, accommodate a product in dry powder form whereas the other container accommodates product in liquid, solution or aerosol form.

In one aspect, the first medicament container and the at least one further medicament container are of a type adapted to be used with a medicament dispenser selected from the group consisting of a reservoir dry powder inhaler (RDPI), a multi-dose dry powder inhaler (MDPI), a metered dose inhaler (MDI) and a liquid spray inhaler (LSI). The first medicament dispenser and at least one further remain different in type.

In one aspect, the first medicament dispenser is a reservoir dry powder inhaler (RDPI), and the at least one further medicament dispenser is of a type selected from the group consisting of a multi-dose dry powder inhaler (MDPI), a metered dose inhaler (MDI) and a liquid spray inhaler (LSI).

In another aspect, the first medicament dispenser is a multi-dose dry powder inhaler (MDPI), and the at least one further medicament dispenser is of a type selected from the group consisting of a reservoir dry powder inhaler (RDPI), a metered dose inhaler (MDI) and a liquid spray inhaler (LSI).

In a further aspect, the first medicament dispenser is a metered dose inhaler (MDI), and the at least one further medicament dispenser is of a type selected from the group consisting of a reservoir dry powder inhaler (RDPI), a multi-dose dry powder inhaler (MDPI) and a liquid spray inhaler (LSI).

In a further aspect, the first medicament dispenser is a liquid spray inhaler (LSI), and the at least one further medicament dispenser is of a type selected from the group consisting of a reservoir dry powder inhaler (RDPI), a multi-dose dry powder inhaler (MDPI) and a metered dose inhaler (MDI).

By reservoir dry powder inhaler (RDPI) it is meant an inhaler having a reservoir form container pack suitable for containing multiple (un-metered doses) of medicament product in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose container pack containing (or otherwise carrying) multiple, define doses (or parts thereof) of medicament product. In a preferred aspect, the carrier has a blister pack form, but it could also, for example, comprise a capsule-based pack form or a carrier onto which medicament has been applied by any suitable process including printing, painting and vacuum occlusion.

In one aspect, the multi-dose pack is a blister pack comprising multiple blisters for containment of medicament product in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 in the name of Glaxo Group Ltd describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085.

By liquid spray inhaler (LSI) it is meant a medicament dispenser suitable for dispensing medicament in spray form, wherein the medicament is typically formulated in liquid or solution form and comprised in a liquid container. The container is typically provided with a means of metering to a spray generator, which imparts energy to the liquid or solution, thereby generating a spray for inhalation by the patient. The spray generator, in aspects, comprises a vibrating element (e.g. a mesh) that provides vibrational energy to the formulation, thereby resulting in its aerosolisation. In other aspects, the spray generator comprises a pump mechanism, which either delivers the medicament directly to the patient (as a liquid spray) or which delivers the medicament to an intermediate position at which further energy is supplied thereto to further propel, aerosolise or otherwise direct the medicament dose to the patient.

In aspects, the first release means and the at least one further release means may either be independently operable or operable in coupled fashion.

The medicament dispenser device herein has unitary form, and typically has a housing shaped to receive, and enable the release of medicament product from the first and at least one further medicament containers.

In one aspect, the housing integrally comprises a release means for releasing medicament from at least one, preferably all of the medicament dispensers. Suitably, the release means for each medicament container is coupled, thereby enabling simultaneous delivery of medicament from each dispenser in response to a single patient actuation step.

In another aspect, the housing is shaped to receive the medicament containers, each of which is provided with respective release means. In this case, the release means have typically been adapted for receipt by the housing. The medicament dispenser and release means therefor are in one aspect, supplied as independently operable ‘cassette refills’ for the unitary device.

The medicament dispensing system comprises a first and at least one further medicament container, each associated with release means for releasing a quantity (e.g. volume or mass) of medicament in response to the electronic control system.

In another aspect, the quantity of medicament to be dispensed is set manually by the patient responsive to dose guidance (e.g. determined by an electronic control system) and indicated to the patient (e.g. visually, on an electronic display).

In one aspect, the quantity of medicament for dispensing is metered from a reservoir of medicament (e.g. in powder or fluid form) by use of any suitable metering means.

Suitably, the meter comprises a valve (for example, a linear or rotary valve) and/or a piston and/or a load cell. In another aspect, the meter comprises a plunger, such as might exist in a syringe.

Suitably, the meter comprises at least one metering cavity or chamber. In one embodiment, the or each metering chamber is reversibly moveable into fluid communication with the reservoir for metering therefrom.

In one embodiment, the meter and the reservoir are relatively rotatable with respect to each other about a common central axis. Preferably, the or each metering cavity or chamber is adapted to be in fluid communication selectively with the reservoir or with the patient.

The or each metering cavity or chamber may have a variable volume. Altematively, the or each metering cavity or chamber may have a fixed volume which is variable by insertion of a plunger or piston. The or each metering cavity or chamber may be formed from expandable material and/or have a telescopic or concertina arrangement.

In one aspect, the dispenser is provided with mixing means for ensuring mixing of the delivered medicaments prior to their delivery to the patient (e.g. by inhalation) as a ‘mixed’ multi-active combination product.

Suitably, the mixing means comprises a mixing chamber including inlets for receiving medicament form each medicament container and an outlet for delivery of ‘mixed’ medicament product to the patient for inhalation (e.g. through a mouthpiece which communicates with the mixing chamber). The ergonomics of the mixing chamber will be arranged to ensure effective mixing of the separate medicament feeds. In aspects, baffles, propellers, venturi and other features for controlling mixing dynamics are provided. The mixing chamber may also be provided with energisation means for energising the mixing process, or alternatively features may be provided to harness the energy provided by a patient's inward breath to enhance the mixing process.

The dispenser device may be provided with means for varying the amount of medicament product released from each medicament container. Customized delivery of combination medicament product may therefore be achieved through varying the relative ratios of each individual medicament product delivered as well as by varying the absolute amount of medicament product delivered. Variable timing mechanisms are envisaged for achieving such customisation.

Delivery of the combination product (e.g. after mixing) to the patient is preferably through a single outlet. The outlet is typically positioned to be in communication with the distinct medicament dose portions delivered. The outlet may have any suitable form. In one aspect, it has the form of a mouthpiece and in another, it has the form of a nozzle for insertion into the nasal cavity of a patient.

The outlet is preferably a single outlet, which communicates with the distinct medicament dose portions delivered via a common air channelling means (e.g. formed as an air-pipe or common manifold). The patient may therefore breathe in through a single outlet, and that breath be transferred through the common channelling means to (all of) the released medicament dose portions, thereby enabling their inhalation as a multi-active combined product.

In addition to, or as an alternative to, any separate mixing chamber, the outlet and/or channelling means may be shaped to encourage mixing of medicament as a result of the air flow created by inhalation by the patient. For example, baffles or other mechanical aids to mixing may be incorporated. Venturi channelling of the air flow is also envisaged in embodiments. Helical form channels are envisaged.

Any or all mechanical components of the device may be driven by either an electronic or mechanical drive system or combination thereof.

Suitably electronic drive means typically comprise a motor, preferably an electrically-powered motor. The motor may provide linear or rotary drive, but in general, rotary motors are most suitable. The motor may for example, comprise a DC electric motor, a piezoelectric (PZ) motor, an ultrasonic motor, a solenoid motor or a linear motor. Preferably, the electronic drive system comprises a DC motor, a PZ motor or an ultrasonic motor.

The use of ultrasonic motors is particularly preferred since they offer advantages over conventional motors in terms of weight, size, noise, cost and torque generated. Ultrasonic motors are well known in the art and are commercially available (e.g. BMSTU Technological Cooperation Centre Ltd, Moscow, Russia; Shinsei Corporation, Tokyo, Japan).

Ultrasonic motors do not use coils or magnets but comprise a piezoelectric ceramic stator which drives a coupled rotor. The stator generates ultrasonic vibrations which in turn causes rotation of the rotor. While regular DC motors are characterised by high speed and low torque, requiring reduction gearing to increase torque, ultrasonic motors attain low speed and high torque, thus eliminating the need for reduction gearing. Furthermore, these motors are lightweight and compact, lacking coils and magnets, and are noiseless as the ultrasonic frequencies used are not audible to the human ear.

Suitably, the device further comprises actuating means for actuating said electronic drive system. Said actuating means may take the form of a switch, push-button, or lever.

In one aspect, the medicament dispenser includes an electronic control system for controlling the release of contents from the first and at least one further medicament container. The electronic control system may have any suitable form and incorporate any of the electronic system aspects as described hereinafter.

In one aspect, the electronic control system is responsive to inputs directly provided to it by an individual such as for example, a medical professional (e.g. G.P.), a pharmacist or the patient. In this aspect, any tailoring of the composition of the combination product is determined by these inputs. In one particular aspect, the inputs are set (or even, pre-set) at particular time such as at the prescription of the dispenser to the patient.

In another aspect, the electronic control system is associated with or responsive to a patient diagnostic system that collects diagnostic information relating to the patent's current disease condition. Tailoring of the composition of the combination product is therefore determinable by reference to diagnostic data gathered and processed by this system.

Where the dispenser is an inhaler for dispensing medicament for the relief of respiratory disorders, examples of suitable diagnostic data would include diagnostics related to the patient's physical breath characteristics including particularly breath cycle data or peak flow or FEV-1 data.

Suitably, there is provided an electronic data management system that is either separate from, integral with or in communication with the electronic control system. The electronic data management system typically has input/output capability and comprises a memory for storage of data; a microprocessor for performing operations on said data; and a transmitter for transmitting a signal relating to the data or the outcome of an operation on the data.

Suitably, the electronic data management system is arranged to be responsive to or activated by the voice of a user. Thus, for example the system may be switched on or off in response to a voice command.

The electronic data management system may be integral with the body. Alternatively, the electronic data management system forms part of a base unit which is reversibly associable with the body.

Suitably, the device additionally comprises a data input system for user input of data to the electronic data management system. Preferably, the data input system comprises a man machine interface (MMI) preferably selected from a keypad, voice recognition interface, graphical user interface (GUI) or biometrics interface.

Energy may be conserved by a variety of means to enable the device to operate for longer on a given source of energy, such as a battery. Energy conservation or saving methods have additional advantages in terms of reducing the size requirements of the power source (e.g. battery) and thus the weight and portability of the medicament dispenser.

A variety of energy saving methods is available which generally involve reducing power consumption. One such method is to use a clock or timer circuit to switch the power on and off at regular or predetermined intervals. In another method the system can selectively switch on/off specific electronic devices, such as visual display units or sensors, in order to power these devices only when they are required to perform a particular sequence of events. Thus different electronic devices may be switched on and off at varying intervals and for varying periods under control of the system. The power sequencing system may also respond to a sensor, such as a motion or breath sensor, which is activated on use of the device.

Low power or “micropower” components should be used within the electronics where possible and if a high power device is required for a particular function this should be put into a low power standby mode or switched off when not required. Similar considerations apply in the selection of transducers. Operation at low voltage is desirable since power dissipation generally increases with voltage.

For low power digital applications complementary metal oxide semi-conductor (CMOS) devices are generally preferred and these may be specially selected by screening for low quiescent currents. Clock speeds of processors and other logic circuits should be reduced to the minimum required for computational throughput as power consumption increases with frequency. Supply voltages should also be kept at minimal values consistent with reliable operation because power dissipation in charging internal capacitance's during switching is proportional to the square of the voltage. Where possible, supply voltages should be approximately the same throughout the circuit to prevent current flowing through input protection circuits. Logic inputs should not be left floating and circuits should be arranged so that power consumption is minimised in the most usual logic output state. Slow logic transitions are undesirable because they can result in relatively large class-A currents flowing. Resistors may be incorporated in the power supply to individual devices in order to minimise current in the event of failure.

In some control applications, devices that switch between on and off states are preferred to those that allow analog (e.g. linear) control because less power is dissipated in low resistance on states and low current off states. Where linear components are used (e.g. certain types of voltage regulators) then types with low quiescent currents should be selected. In some circuit configurations it is preferable to use appropriate reactive components (i.e. inductors and capacitors) to reduce power dissipation in resistive components.

Suitably, the system additionally comprises a visual display unit for display of data from the electronic data management system to the user. The display may for example, comprise a screen such as an LED or LCD screen. More preferably the visual display unit is associable with the body of the medicament dispenser.

Suitably, the device additionally comprises a datalink for linking to a local data store to enable communication of data between the local data store and the electronic data management system. The datastore may also comprise data management, data analysis and data communication capability.

The datastore may itself form part of a portable device (e.g. a handheld device) or it may be sized and shaped to be accommodated within the patient's home. The datastore may also comprise a physical storage area for storage of replacement cassettes. The datastore may further comprise a system for refilling medicament from a reservoir of medicament product stored therewithin. The datastore may further comprise an electrical recharging system for recharging any electrical energy store on the medicament dispenser, particularly a battery recharging system.

The datalink may for example enable linking with a docking station, a personal computer, a network computer system or a set-top box by any suitable method including a hard-wired link, an infrared link or any other suitable wireless communications link.

In one aspect, the device includes an electronic dose reminder system. This may be configured to have any suitable form and may be powered by a mains, stored (e.g. battery) or self-regenerating (e.g. solar) energy power source.

The electronic dose reminder system comprises an electronic timer for timing an elapsed time period corresponding to the time since the last actuation of the device; a dose interval memory for storing data relating to a prescribed dose interval time period; and a patient alerter for alerting a user. The alerter activates when the elapsed time period exceeds the prescribed dose interval time period.

The electronic timer progressively times the period since the last actuation of the device (the ‘elapsed time period’). The timer can have any suitable electronic form. The significance of the ‘elapsed time period’ is that in use, it typically corresponds to the time elapsed since the previous dose delivery event.

The timer may be configured to include an automatic re-zeroing feature such that on subsequent actuation of the device the timer count starts again from zero.

The dose interval memory stores data relating to a prescribed dose interval time period. By way of examples, if the medicament is to be taken twice a day at a regular interval, the prescribed dose interval may be set as twelve hours, or for a once daily treatment the value may be set at twenty four hours. In aspects, the system may be configured to allow for ready readjustment of the prescribed dose interval time period, or it may be configured in secure fashion such that any readjustment may be made only by a designated prescriber (e.g. a medical professional or pharmacist). Password and/or other security means may be employed. The prescribed dose interval may be configured to be variable over a particular course of treatment, or alternatively it may be fixed at a set dose interval over the full course of treatment.

The patient alerter is designed to communicate an alert to the user. The alerter activates only when the holding time period exceeds the prescribed dose interval time period. By way of an example, for a once daily treatment with a prescribed dose interval of twenty four hours, the alerter would activate only when the holding time period, as timed by the electronic timer, exceeds twenty four hours since at this point another dose is due to be taken. It may thus, be appreciated that the alerter acts functionally as a reminder to the patient that a dose is due to be taken.

The alerter may in aspects, comprise a visual device, such as a liquid crystal display (LCD) or an array of light-emitting diodes (LEDs), connected to a battery-driven timing device of any convenient kind known to those skilled in the art. The visual device may be configured to display information such as the actual time or the elapsed time from the taking of a previous dosage and may have superimposed thereon additional messages, such as a textual instruction to take a dose of the medicament. Alternatively, the instruction to take the medicament may be conveyed merely by displaying a warning colour or by causing the display to flash or in any other way.

In a further alternative arrangement, no specific time or elapsed time information is displayed, but the alerter merely provides a warning signal that indicates the necessary action to the user.

Depending upon the lifestyle of the user, additional or alternative warnings may be of greater assistance than purely visual warnings. Accordingly, the invention envisages that the alerter may provide audible and/or tactile warnings, such as vibration, instead of (or in addition to) visual warnings.

The alerter may provide a single, one-off alert. More preferably, the alerter is configured to provide the alert over a set period of time (the ‘alerting time period’ or ‘alerting window’). In one aspect, the alerting time period is calculated as a function of (e.g. fraction of) the dose interval time period. For example, for a twice daily treatment with a dose interval time period of twelve hours, the alerting time period may be set as half that period (i.e. six hours). In this case, the alert is then provided for the six hours immediately following the activation of the alert.

The system is typically configured such that the alerting signal cuts off when the user removes the medicament delivery device from the holder to enable dosing of medicament therefrom. The system is then reset. Other manual cutoffs/overrides may also be included.

It may be appreciated that the relevant timeframe for detecting, timing and alerting are determined by user action in relation to the system, and in particular by user action. The dose reminder capability is therefore suitably independent of any particular defined external time zone (e.g. the local time zone relative to Greenwich Mean Time, as defined by the twenty four hour clock) because the user action defines its own ‘reminder timeframe’. This provides advantages over other known reminder systems, which are reliant on user reference to defined external time frames. The advantage is particularly great for the international traveller since complex calculations involving different local time zones are avoided.

It will be appreciated from the above description that the various components of the electronic dose reminder system interrelate with each other to provide the required functionality. The system may be configured in any suitable fashion using known electronic components and circuitry methods.

Suitably, the device additionally comprises an actuation detector for detecting actuation of any one of the medicament dispensers thereof wherein said actuation detector transmits actuation data to the electronic data management system.

The device may additionally comprise a safety mechanism to prevent unintended multiple actuations of the component medicament dispensers. The patient is thereby, for example, protected from inadvertently receiving multiple doses of medicament in a situation where they take a number of short rapid breaths. More preferably, the safety mechanism imposes a time delay between successive actuations of the release means. The time delay is typically of the order of from three to thirty seconds.

Suitably, the device additionally comprises a release detector for detecting release of medicament from the cassette, wherein said release detector transmits release data to the electronic data management system.

Suitably, the device additionally comprises a shake detector for detecting shaking of the medicament container (e.g. prior to actuation of the dispensing mechanism), wherein said shake detector transmits shake data to the electronic data management system.

Suitably, any actuation detector, release detector, or shake detector comprises a sensor for detecting any suitable parameter such as movement. Any suitable sensors are envisaged including the use of optical sensors. The release detector may sense any parameter affected by release of the medicament such as pressure, temperature, sound, moisture, carbon dioxide concentration and oxygen concentration.

Suitably, the medicament dispenser is actuable in response to the inward breath of a patient and includes a breath sensor of any suitable type (e.g. mechanical or electronic) for detecting that inward breath wherein the sensor communicates with the electronic control system. Thus, in use the patient breathes in through the dispenser (e.g. through the mouthpiece); the breath is detected by the breath sensor; the sensor communicates with the electronic control system to convey an ‘inward breath detected’ signal; and the electronic control system responds by releasing medicament from one or more of the medicament containers for inhalation by the patient.

In one aspect, the breath sensor comprises a breath-movable element that is movable in response to the breath of a patient. Preferably, the breath-movable element is selected from the group consisting of a vane, a sail, a piston and an impeller.

In another aspect, the breath sensor comprises a pressure sensor for sensing the pressure profile associated with the breath of a patient.

In a further aspect, the breath sensor comprises an airflow sensor for sensing the airflow profile associated with the breath of a patient.

In a further aspect, the breath sensor comprises a temperature sensor for sensing the temperature profile associated with the breath of a patient.

In a further aspect, the breath sensor comprises a moisture sensor for sensing the moisture profile associated with the breath of a patient.

In a further aspect, the breath sensor comprises a gas sensor for sensing the oxygen or carbon dioxide profile associated with the breath of a patient.

In a further aspect, the breath sensor comprises a piezoelectric or piezoresistive element.

In one aspect, the dispenser additionally comprises a breath-responsive trigger for triggering one or all of the component medicament dispensers, said breath-responsive trigger being actuable in response to a trigger signal from the electronic control system or electronic data management system. Suitably, the electronic data management system includes a predictive algorithm or look-up table for deriving from the breath data when to transmit the trigger signal. For example, a real-time analysis of the patient breath waveform may be made and the trigger point derived by reference to that analysed waveform.

In one aspect, the medicament dispenser herein includes a timing control system for controlling the time of release of contents from the first and at least one further medicament container. The timing control system generally communicates with the electronic control system with which it may in aspects, form an integral part.

The timing control system is suitably arranged to vary the relative time of release of each medicament component from its respective medicament container. Each medicament component may therefore be arranged for simultaneous or sequential release, although in general where components are released sequentially the time delay between releases of each separate medicament component is short (e.g. milliseconds) to ensure that a combined product is provided for administration to the patient.

In a further aspect, by varying the time of release, the ratio of quantity of each medicament component released can also be varied, thereby enabling the provision and delivery of ‘tailored’ combined products.

The timing control system generally comprises electronic components and is arranged to be responsive to the electronic control system. In aspects, the timing control system is arranged to be responsive to a diagnostic system, which is arranged to diagnose patient disease characteristics and thereby select and deliver and suitable tailored combined product dose.

Suitably, the electronic data management system includes a predictive algorithm or look-up table for calculating the optimum amount of medicament to dispense.

Suitably, the memory on the electronic data management system includes a dose memory for storing dosage data and reference is made to the dose memory in calculating the optimum amount of medicament to dispense.

Suitably, the device additionally comprises a selector for selecting the amount of medicament to dispense from said dispensing mechanism. In one aspect, the selector is manually operable. In another aspect, the selector is operable in response to a signal from the transmitter on the electronic data management system.

Suitably, the device comprises in association with a body or housing thereof, a first transceiver for transmitting and receiving data and in association with the medicament container, a second transceiver for transmitting and receiving data, wherein data is transferable in two-way fashion from the first transceiver to the second transceiver. The data is preferably in digital form and suitable for transfer by electronic or optical means. A medicament dispenser of this general type is described in pending UK Patent Application No. 0020538.5.

One advantage of embodiments of this type is the ability to store many types of information in different parts of the memory structure of the transceivers. The information is furthermore stored in a form which is readily and accurately transferable. The information could for example, include manufacturing and distribution compliance information written to the, memory at various points in the manufacturing or distribution process, thereby providing a detailed and readily accessible product history of the dispenser. Such product history information may, for example, be referred to in the event of a product recall. The compliance information could, for example, include date and time stamps. The information could also include a unique serial number stored in encrypted form or in a password protectable part of the memory which uniquely identifies the product and therefore may assist in the detection and prevention of counterfeiting. The information could also include basic product information such as the nature of the medicament and dosing information, customer information such as the name of the intended customer, and distribution information such as the intended product destination.

On loading or reloading the device with a medicament dispenser or ‘refill’ the second transceiver may, for example, read the unique serial number, batch code and expiry date of the medicament and any other information on the second transceiver. In this way the nature and concentration of the medicament, together with the number of doses used or remaining within the cassette, may be determined. This information can be displayed to the patient on a visual display unit. Other information, such as the number of times the medicament dispenser has been reloaded with a cassette, may also be displayed.

Similarly, should the cassette be removed from the holder before the supply of medicament is exhausted, the same data can be read from the second transceiver and the number of doses remaining or used determined. Other information, such as the date and time of administration of the drug, or environmental exposure data such as the minimum/maximum temperatures or levels of humidity the cassette has been exposed to, may also be read and displayed to the user.

In the event that the supply of medicament within any medicament container becomes exhausted, or that the shelf life of the medicament has expired, or that the first transceiver does not recognise the batch code on the second transceiver, activation of the dispenser may be prevented to safeguard the user. Activation may also be prevented if the medicament has been exposed to extreme environmental conditions for periods outwith the manufacturer's guidelines.

Data may be transferred to and from any transceiver during the period of use of the medicament dispenser by the patient. For example, the medicament dispenser may include an electronic data management system having various sensors associated therewith. Any data collected by the sensors or from any data collection system associated with the electronic data management system including a clock or other date/time recorder is transferable.

Data may be transferred each time the patient uses the device. Or alternatively, data may be stored in a database memory of the electronic data management system and periodically downloaded to any transceiver. In either case, a history of the usage of the device may be built up in the memory of a transceiver.

In one embodiment herein, a history of the usage of the device is transferred to the second transceiver. When the medicament carriers in the cassette are exhausted it is exchanged by the patient for a new refill cassette. At the point of exchange, which will typically occur at the pharmacy, data may be transferred from the exhausted cassette to the refill and vice-versa. Additionally, usage history data may be read from the refill and transferred to a healthcare data management system for example comprising a network computer system under the control of a healthcare data manager.

Methods are envisaged herein whereby the patient is given some sort of reward for returning the refill and making available the data comprised within the second transceiver. Methods are also envisaged herein whereby the healthcare data manager is charged for either receipt of the data from the second transceiver or for its use for commercial purposes. Any rewards or charging may be arranged electronically. The methods may be enabled by distributed or web-based computer network systems in which any collected data is accessible through a hub on the network. The hub may incorporate various security features to ensure patient confidentiality and to allow selective access to information collected dependent upon level of authorisation. The level of user authorisation may be allocated primarily to safeguard patient confidentiality. Beyond this the level of user authorisation may also be allocated on commercial terms with for example broader access to the database being authorised in return for larger commercial payments.

Suitably, the first and second transceiver each comprise an antenna or equivalent for transmitting or receiving data and connecting thereto a memory. The memory will typically comprise an integrated circuit chip. Either transceiver may be configured to have a memory structure which allows for large amounts of information to be stored thereon. The memory structure can be arranged such that parts of the memory are read-only, being programmed during/after manufacture, other parts are read/write and further parts are password protectable. Initial transfer of information (e.g. on manufacture or one dispensing) to or from any transceiver can be arranged to be readily achievable by the use of a reader which is remote from the medicament dispenser, thereby minimising the need for direct product handling. In further aspects, the reader can be arranged to simultaneously read or write to the memory of multiple transceivers on multiple medicament dispensers.

A suitable power source such as a battery, clockwork energy store, solar cell, fuel cell or kinetics-driven cell will be provided as required to any electronic component herein. The power source may be arranged to be rechargeable or reloadable.

Suitably, data is transferable in two-way fashion between the first and second transceiver without the need for direct physical contact therebetween. Preferably, data is transferable wirelessly between the first and second transceiver.

Suitably, the first transceiver is an active transceiver and the second transceiver is a passive transceiver. The term active is used to mean directly-powered and the term passive is used to mean indirectly-powered.

Suitably, the second transceiver comprises a label or tag comprising an antenna for transmitting or receiving energy; and an integrated circuit chip connecting with said antenna, and the first transceiver comprises a reader for said label or tag. In this case the label or tag is a passive transceiver and the reader is an active transceiver. Preferably, the reader will not need to be in direct contact with the tag or label to enable the tag or label to be read.

The tag may be used in combination and/or integrated with other traditional product labelling methods including visual text, machine-readable text, bar codes and dot codes.

Suitably, the integrated circuit chip has a read only memory area, a write only memory area, a read/write memory area or combinations thereof.

Suitably, the integrated circuit chip has a one-time programmable memory area. More preferably, the one-time programmable memory area contains a unique serial number.

Suitably, the integrated circuit chip has a preset memory area containing a factory preset, non-changeable, unique data item. The preset memory item is most preferably in encrypted form.

Suitably, the integrated circuit chip has plural memory areas thereon. Suitably, any memory area is password protected.

Suitably, any memory area contains data in encrypted form. Electronic methods of checking identity, error detection and data transfer may also be employed.

In one aspect, the integrated circuit has plural memory areas thereon including a read only memory area containing a unique serial number, which may for example be embedded at the time of manufacture; a read/write memory area which can be made read only once information has been written thereto; and a password protected memory area containing data in encrypted form which data may be of anti-counterfeiting utility.

Suitably, the tag is on a carrier and the carrier is mountable on the body or holder of the medicament dispenser or on the cassette.

In one aspect, the carrier is a flexible label. In another aspect, the carrier is a rigid disc. In a further aspect, the carrier is a rectangular block. In a further aspect, the carrier is a collar ring suitable for mounting to the neck of an aerosol container. Other shapes of carrier are also envisaged.

Suitably, the carrier is mouldable or weldable to the cassette or housing. Suitably, the carrier encases the tag. More preferably, the carrier forms a hermetic seal for the tag.

In one aspect, the carrier comprises an insulating material such as a glass material or, a paper material or an organic polymeric material such as polypropylene. Alternatively, the carrier comprises a ferrite material.

The energy may be in any suitable form including ultrasonic, infrared, radiofrequency, magnetic, optical and laser form. Any suitable channels may be used to channel the energy including fibre optic channels.

In one aspect, the second transceiver comprises a radiofrequency identifier comprising an antenna for transmitting or receiving radiofrequency energy; and an integrated circuit chip connecting with said antenna, and the first transceiver comprises a reader for said radiofrequency identifier. In this case the radiofrequency identifier is a passive transceiver and the reader is an active transceiver. An advantage of radiofrequency identifier technology is that the reader need not be in direct contact with the radiofrequency identifier tag or label to be read.

The radiofrequency identifier can be any known radiofrequency identifier. Such identifiers are sometimes known as radiofrequency transponders or radiofrequency identification (RFID) tags or labels. Suitable radiofrequency identifiers include those sold by Phillips Semiconductors of the Netherlands under the trade marks Hitag and Icode, those sold by Amtech Systems Corporation of the United States of America under the trade mark Intellitag, and those sold by Texas Instruments of the United States of America under the trade mark Tagit.

Suitably, the antenna of the RFID tag is capable of transmitting or receiving radiofrequency energy having a frequency of from 100 kHz to 2.5 GHz. Preferred operating frequencies are selected from 125 kHz, 13.56 MHz and 2.4 GHz.

In one aspect, the second transceiver comprises a magnetic label or tag comprising an antenna for transmitting or receiving magnetic field energy; and an integrated circuit chip connecting with said antenna, and the first transceiver comprises a reader for said magnetic label or tag. In this case the magnetic label or tag is a passive transceiver and the reader is an active transceiver.

A suitable magnetic label or tag comprises plural magnetic elements in mutual association whereby the magnetic elements move relative to each other in response to an interrogating magnetic field. A magnetic label or tag of this type is described in U.S. Pat. No. 4,940,966. Another suitable magnetic label or tag comprises a magnetorestrictive element which is readable by application of an interrogating alternating magnetic field in the presence of a magnetic bias field which results in resonance of the magnetorestrictive elements at different predetermined frequencies. A magnetic label of this type is described in PCT Patent Application No. WO92/12402. Another suitable magnetic label or tag comprising plural discrete magnetically active regions in a linear array is described in PCT Patent Application No. WO96/31790. Suitable magnetic labels and tags include those making use of Programmable Magnetic Resonance (PMR) (trade name) technology.

In another aspect, the second transceiver comprises a microelectronic memory chip and the first transceiver comprises a reader for said microelectronic memory chip. The microelectronic memory chip may comprise an Electrically Erasable Programmable Read Only Memory (EEPROM) chip or a SIM card-type memory chip. In this case the microelectronic memory chip is a passive transceiver and the reader is an active transceiver.

Any transceiver herein, particularly a passive transceiver may be mounted on or encased within any suitable inert carrier. The carrier may comprise a flexible sheet which may in embodiments be capable of receiving printed text thereon.

In one aspect, the first transceiver is integral with the body such that a single unit is comprised. The first transceiver may for example be encased within or moulded to the body.

In another aspect, the first transceiver forms part of a base unit which is reversibly associable with the body. The base unit may for example, form a module receivable by the body such as a snap-in module.

Suitably, the device additionally comprises a communicator for wireless communication with a network computer system to enable transfer of data between the network computer system and the electronic data management system. Dispensers employing such communicators are described in pending PCT Applications No.s PCT/EP00/09291 (PG3786), PCT/EP00/09293 (PG4029) and PCT/EP00/09292 (PG4159). Preferably, the communicator enables two-way transfer of data between the network computer, system and the electronic data management system.

Suitably, the data is communicable between the network computer system and the electronic data management system in encrypted form. All suitable methods of encryption or partial encryption are envisaged. Password protection may also be employed. Suitably, the communicator employs radiofrequency or optical signals.

In one aspect, the communicator communicates via a gateway to the network computer system. In another aspect, the communicator includes a network server (e.g. a web server) such that it may directly communicate with the network.

In a further aspect, the communicator communicates with the gateway via a second communications device. Preferably, the second communications device is a telecommunications device, more preferably a cellular phone or pager. Preferably, the communicator communicates with the second communications device using spread spectrum radiofrequency signals. A suitable spread spectrum protocol is the Bluetooth (trade mark) standard which employs rapid (e.g. 1600 times a second) hopping between plural frequencies (e.g. 79 different frequencies). The protocol may further employ multiple sending of data bits (e.g. sending in triplicate) to reduce interference.

In one aspect, the network computer system comprises a public access network computer system. The Internet is one suitable example of a public access network computer system, wherein the point of access thereto can be any suitable entrypoint including an entrypoint managed by an Internet service provider. The public access network computer system may also form part of a telecommunications system, which may itself be either a traditional copper wire system, a cellular system or an optical network.

In another aspect, the network computer system comprises a private access network computer system. The private access network system may for example, comprise an Intranet or Extranet which may for example, be maintained by a health service provider or medicament manufacturer. The network may for example include password protection; a firewall; and suitable encryption means.

Preferably, the communicator enables communication with a user-specific network address in the network computer system.

The user-specific network address may be selected from the group consisting of a web-site address, an e-mail address and a file transfer protocol address. Preferably, the user-specific network address is accessible to a remote information source such that information from said remote information source can be made available thereto. More preferably, information from the user-specific network address can be made available to the remote information source.

In one aspect, the remote information source is a medicament prescriber, for example a doctors practice. Information transferred from the medicament prescriber may thus, comprise changes to prescription details, automatic prescription updates or training information. Information transferred to the medicament prescriber may comprise compliance information, that is to say information relating to the patient's compliance with a set prescribing programme. Patient performance information relating for example, to patient-collected diagnostic data may also be transferred to the medicament prescriber. Where the dispenser is an inhaler for dispensing medicament for the relief of respiratory disorders examples of such diagnostic data would include breath cycle data or peak flow data.

In another aspect, the remote information source is a pharmacy. Information transferred from the pharmacy may thus, comprise information relating to the medicament product. Information sent to the pharmacy may thus include prescription requests which have been remotely pre-authorised by the medicament prescriber.

In a further aspect, the remote information source is an emergency assistance provider, for example a hospital accident and emergency service or an emergency helpline or switchboard. The information may thus, comprise a distress or emergency assist signal which requests emergency assistance.

In a further aspect, the remote information source is a manufacturer of medicament or medicament delivery systems. Information transferred to the system may thus, comprise product update information. The system may also be configured to feed information back to the manufacturer relating to system performance.

In a further aspect, the remote information source is a research establishment. In a clinical trial situation, information may thus be transferred relating to the trial protocol and information relating to patient compliance fed back to the research establishment.

In a further aspect, the remote information source is an environmental monitoring station. Information relating to weather, pollen counts and pollution levels may thus be made accessible to the system.

Suitably, the device additionally comprises a geographic positioning system such as a global positioning system or a system which relies on the use of multiple communications signals and a triangulation algorithm.

The constituent medicaments of the plural medicament dose portions suitably, in combination comprise a combination medicament product. Suitably the medicaments are selected from the group consisting of albuterol, salmeterol, fluticasone propionate and beclomethasone dipropionate and salts or solvates thereof. Preferably, the combination comprises salmeterol xinafoate and fluticasone propionate.

### BRIEF DESCRIPTION OF THE DRAWINGS

The invention will now be described with reference to the accompanying drawings in which:

FIG. 1a shows a perspective view of a first dispenser herein;

FIG. 1b shows a rear view of the first dispenser of FIG. 1a;

FIG. 1c shows a sectional side view of the first dispenser of FIGS. 1a and 1b;

FIG. 2a shows a rear view of a medicament container refill set for a second dispenser herein;

FIG. 2b shows a perspective view of the second dispenser incorporating the refill set of FIG. 2a;

FIG. 3a shows a sectional rear view of a refill set for use in a third dispenser herein;

FIG. 3b shows a rear view of the refill set of FIG. 3a;

FIG. 4a shows a sectional side view of a fourth dispenser herein;

FIG. 4b shows a sectional front view of the fourth dispenser of FIG. 4a;

FIG. 5 shows a schematic representation of an electronically-enabled dispensing system herein;

FIGS. 6a to 6c show a reservoir dry powder (RDPI) herein respectively in perspective, exploded (part cut-away) and sectional side views; and

FIG. 7a shows a sectional plan view of a base unit multi-dose dry powder inhaler (MDPI) herein and FIG. 7b illustrates a protective cover unit for mounting to the base unit of FIG. 7a.

### DETAILED DESCRIPTION OF THE DRAWINGS

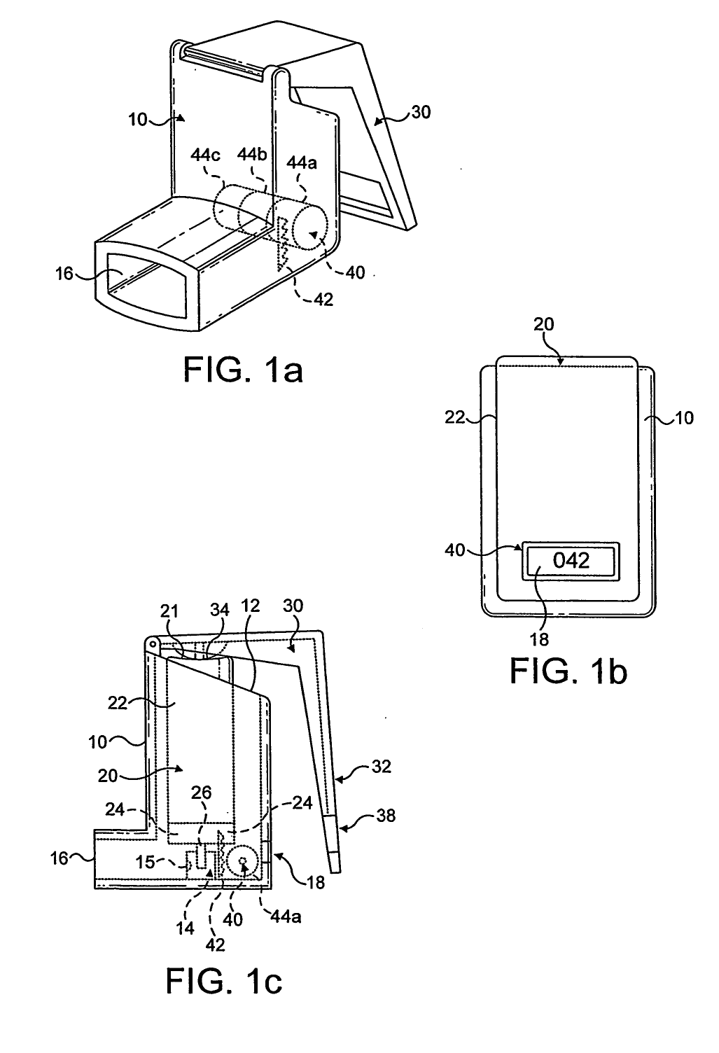

FIGS. 1a to 1c show a first dispenser herein of a metered dose inhaler type. The dispenser comprises an actuator housing 10 of a generally boot-shaped configuration. The actuator housing 10 has a top opening 12 sized and shaped for receipt of plural (only one shown for clarity) aerosol containers 20, each having a container body 22, neck 24 and slide metering valve with valve stem 26. Each valve stem 26 is received by a stem block 14 provided towards the base of the actuator housing, wherein each stem block has a nozzle outlet 15 arranged to direct aerosol spray towards a mouthpiece 16. The actuator housing 10 is further provided with a single hinged actuation lever 30 with handle 32 shaped for thumb depression by a patient and plural pressure pads 34, each of which engages the top 21 of a container body 22.

In operation, patient actuation of the lever 30 transmits downward force through pressure pad 34 to each container body 22, which in turn results in downward force on each slide valve against each stem block 14, which results in the valve being actuated to release aerosol form medicament through the valve stem 26 to the nozzle outlet 15 of the stem block and thence to the mouthpiece 16 for inhalation by the patient. It will thus be appreciated that the patient movement of the single lever 30 results in the near-simultaneous firing and release of aerosol form medicament from each container body 22. Where the various container bodies comprise different medicament types, a combined product is thereby made available for inhalation by the patient.

The actuator housing 10 is also provided with a counter mechanism 40 comprising a single rack drive arm 42 arranged, on actuation, to drivably engage a counter comprising plural, mutually-coupled rotatable drums 44a to 44c, each having numerical indicia thereon (typically capable of counting from 000 to 999 in single count steps). A counter mechanism of this general type is described in more detail in PCT Patent Application No. WO 98/56444 in the name of Glaxo Group Ltd. In use, as container body 22 is depressed following actuation of the lever 30, the rack drive arm 42 drivably engages the counter, thereby causing rotation of one or more of the drums 44a to 44c to result in a counting on of the numerical value displayed by the counter 40. A window 18 is provided to the actuator housing 10 to make the count indicia visible to the user, and a similar window 38 provided to the actuator lever 30 to make the count visible even when the lever 30 is depressed.

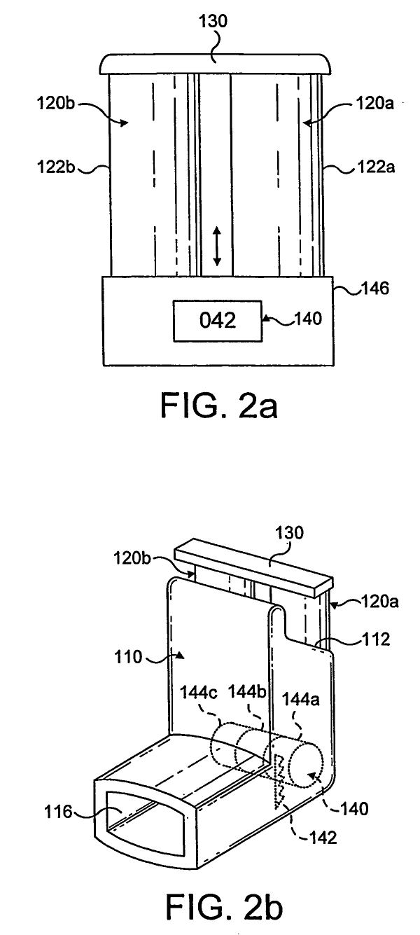

FIGS. 2a to and 2b respectively show a combination refill set and second dispenser for use therewith of the metered dose inhaler type. The dispenser comprises an actuator housing 110 of a generally boot-shaped configuration. The actuator housing 110 has a top opening 112 sized and shaped for receipt of a refill set comprising plural aerosol containers 120a, 120b, each having a container body 122a, 122b, neck and slide metering valve with valve stem (these features not visible). As in FIGS. 1 to 1c, each valve stem will be received by a stem block provided towards the base of the actuator housing, wherein each stem block has a nozzle outlet arranged to direct aerosol spray towards a mouthpiece 116. The respective container bodies 122a, 122b are coupled to each other by top mounted bar 130, which is itself sized and shaped for thumb depression by a patient. The bar 130 may be mounted by any suitable method including snap-fit and heat shrink to fit methods. It will be appreciated that the effect of the coupling bar 130 is to couple together the plural containers 122a, 122b such that it use, their movement is also coupled (e.g. when one is depressed, the other moves with it).

In operation, patient thumb actuation of the coupling bar 130 transmits downward force to the top of each container 120a, 120b, which in turn results in actuation of the valve of each container 120a, 120b to release aerosol form medicament to the mouthpiece 116 for inhalation by the patient. It will thus be appreciated that the patient movement of the single coupling bar 130 results in the near-simultaneous firing and release of aerosol form medicament from each container body 122a, 122b. Where the various container bodies comprise different medicament types, a combined product is thereby made available for inhalation by the patient.

The refill set is also provided with a counter mechanism 140 comprising a single rack drive arm 142 arranged, on actuation, to drivably engage a counter comprising plural, mutually-coupled rotatable drums 144a to 144c, each having numerical indicia thereon (typically capable of counting from 000 to 999 in single count steps). A counter mechanism of this general type is described in more detail in PCT Patent Application No. WO 98/56444 in the name of Glaxo Group Ltd. The counter mechanism 140 is comprised within counter housing 146. Each container 120a, 120b is movable in the housing 146 in general upwards/downwards fashion to enable a count to be registered. That movement would be independent (i.e. each container 120a, 120b separately movable in the counter housing 146) other than for the coupling effect of the coupling bar 130.

In use, as the containers 120a, 120b are depressed following actuation of the coupling bar 130, the rack drive arm 142 drivably engages the counter, thereby causing rotation of one or more of the drums 144a to 144c to result in a counting on of the numerical value displayed by the counter 140. As in the embodiment of FIGS. 1b and 1c, a window would be provided to the actuator housing 110 to make the count indicia visible to the patient through the actuator housing 110.

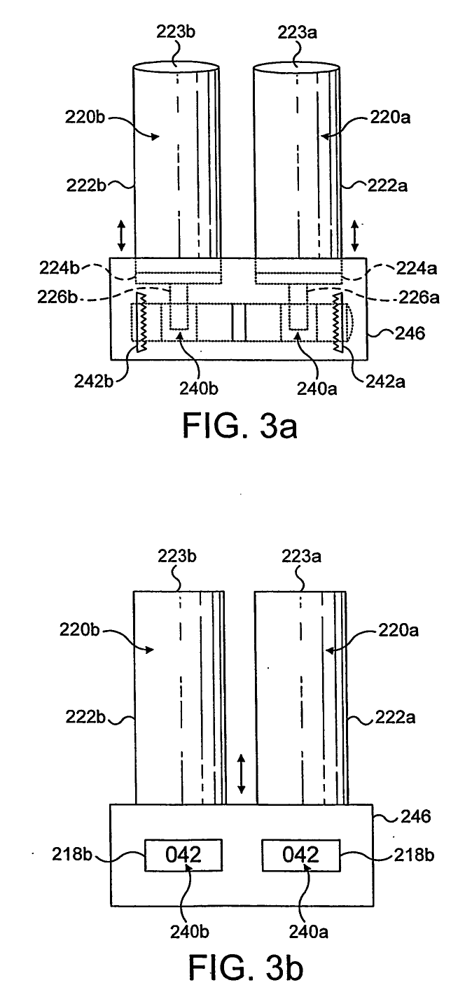

FIGS. 3a and 3b show a refill set for use in a metered dose inhaler type inhaler, particularly one having an open-topped actuator of a generally boot-shaped configuration (e.g. a variation of the actuator of FIGS. 1a or 2b).

The refill set comprises plural aerosol containers 220a, 220b, each having a container body 222a, 222b, neck 224a, 224b and slide metering valve with valve stem 226a, 226b. Once incorporated in a boot actuator (not shown), each valve stem 226a, 226b would be received by a stem block at the base of the actuator housing, the stem block having an outlet arranged to direct aerosol spray towards a mouthpiece of the actuator.

Whilst, the refill set will generally be used with an actuator (as described above), the counting action may be understood by reference to the refill alone. Thus, in operation, the patient transmits to the top 223a, 223b of each container body 22 (e.g. by a two-finger actuating motion). In turn, downward force is transmitted to force each valve stem 226a, 226b against each stem block of the actuator (not shown), which results in the valve being actuated to release aerosol form medicament through the valve stem 226a, 226b to the mouthpiece of the actuator (not shown) for inhalation by the patient. It be appreciated that a two-finger patient actuation would generally result in the near-simultaneous firing and release of aerosol form medicament from each container body 222a, 222b, although the success of this operation is of course, patient-dependent. Where the various container bodies 222a, 222b comprise different medicament types, a combined product is thereby made available for inhalation by the patient.

The refill set is also provided with plural counter mechanisms 240a, 240a, each comprising a single rack drive arm 242a, 242b arranged, on actuation, to drivably engage a counter comprising plural, mutually-coupled rotatable drums (details not labelled on Figures) each having numerical indicia thereon (typically capable of counting from 000 to 999 in single count steps). A counter mechanism of this general type is described in more detail in PCT Patent Application No. WO 98/56444 in the name of Glaxo Group Ltd. The counter mechanisma 240a, 240b are both comprised within counter housing 246. Each container 220a, 220b is independently movable in the housing 246 in general upwards/downwards fashion to enable a count to be registered for each independent container 220a, 220b of the refill.

In use, as each container 222a, 222b is depressed following patient downward actuating force, each rack drive arm 242a, 242b drivably engages each counter 240a, 240b, thereby resulting in a counting on of the numerical value displayed by each counter 240a, 240b. Windows 218a, 218b are provided to the counter housing 246 to make each count indicia visible to the user. This embodiment enables the patient to check that a combined dose has been delivered by making reference to each counter 240a, 240b to ensure that a ‘dose portion’ has been delivered from each container 220a, 220b. This set up has the possible advantage of flexibility, in that a patient may choose to deliver only a part component of a combined product. One disadvantage however, is that a significant degree of patient co-ordination and checking is required to actuate and count the delivery of a combined product.

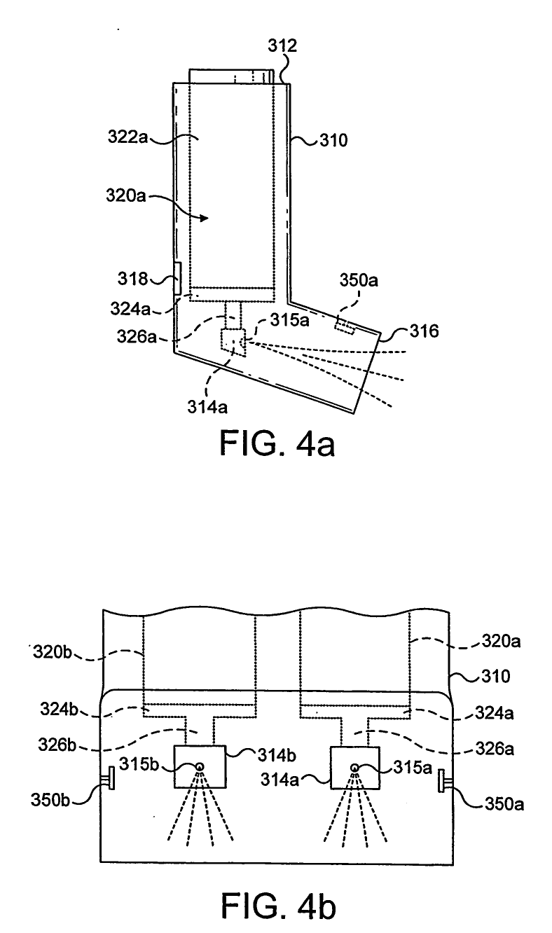

The previous embodiments rely on detecting and registering a count relating to the actuation of the device. The inhaler device shown in FIGS. 4a and 4b instead relies of detection of the release of medicament to register a count.

In more detail, FIGS. 4a and 4b show a dispenser herein of a metered dose inhaler type. The dispenser comprises an actuator housing 310 of a generally boot-shaped configuration. The actuator housing 310 has a top opening 312 sized and shaped for receipt of plural (only one visible in FIG. 4a) aerosol containers 320a, 320b, each having a container body 322a, 322b, neck 324a, 324b and slide metering valve with valve stem 326a, 326b. Each valve stem 326a, 326b is received by a respective stem block 314a, 314b provided towards the base of the actuator housing 310, wherein each stem block has a nozzle outlet 315a, 315b arranged to direct aerosol spray towards a mouthpiece 316.

In operation, patient actuation transmits downward force to each container 320a, 320b (which may be coupled as shown in FIGS. 2a to 2b), which in turn results in downward force on each slide valve 326a, 326b against each stem block 314, which results in the valve being actuated to release aerosol form medicament through the valve stem 326a, 326b to the nozzle outlet 315 of the stem block and thence to the mouthpiece 316 for inhalation by the patient. Desirably, this results in the near-simultaneous firing and release of aerosol form medicament from each container body 322a, 322b. Where the various container bodies comprise different medicament types, a combined product is thereby made available for inhalation by the patient.

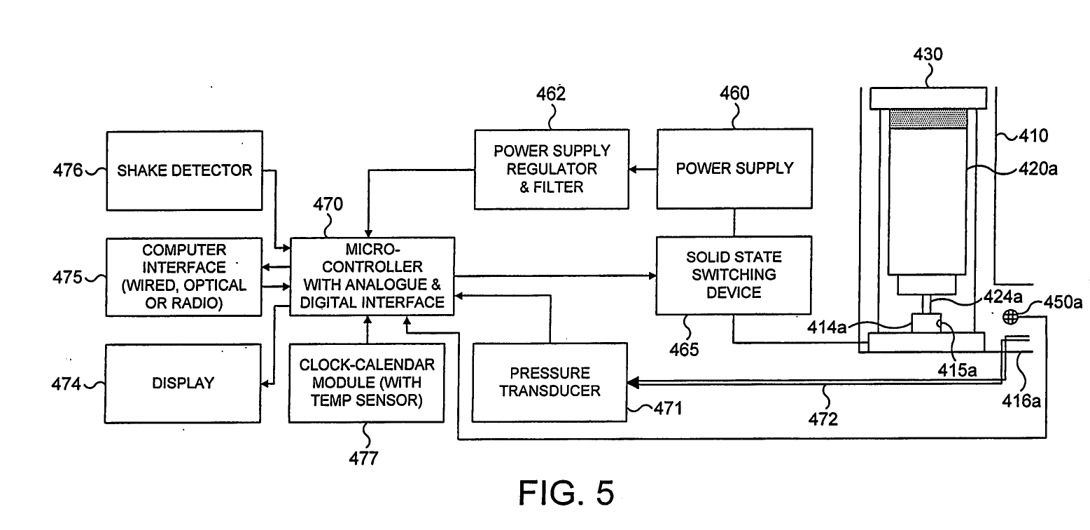

The actuator housing 310 is also provided with plural medicament release sensors 350a, 350b each of which is arranged (e.g. positioned) to separately detect the release of medicament product from each outlet nozzle 315a, 315b of each respective stem block 314a, 314b. Suitable dose release sensors and systems are described in more detail in PCT Patent Application No. WO 02/36190 and the UK Patent Application No. 0209531.3 in the name of Glaxo Group Ltd. Display 318, which communicates with each sensor via electronic sensor processing circuitry (not shown in FIGS. 4a and 4b) is provided to the actuator housing 310 to display release/count data to the patient. In variations, the display may provide data relevant to any of the following: successful detection of release of each medicament separately, or for the combination; and number of doses released or remaining in each container 320a, 320b. Other data may also displayed as described in FIG. 5.

FIG. 5 shows a schematic representation of a dispensing system herein. The system comprises a metered dose inhaler with release sensors similar to that shown in more detail in FIGS. 4a and 4b comprising tubular housing 410 having a dispensing outlet 416 in the form of a mouthpiece. Within the housing 410 sit plural aerosol containers 420a (only one visible) each of which has a valve. For each container 420a, valve stem 424a is supported by valve support 414a. Outlet nozzle 415a is provided in the support 414a to enable passage of dispensed dose to the dispensing outlet 416a. Discrete infrared release sensors 450a (one only shown for clarity) is located in outlet 416a to detect release of medicament from each medicament container 420a. An emitter (not shown) emits an infra red beam (not shown) across outlet 416 on to each relevant sensor 450a.

It may be seen that the upper part each aerosol container 420a abuts actuation coupling bar 430 (similar to that described in FIGS. 2a and 2b). On actuation, the coupling bar is depressed to force each valve stem 424a against its stem block 414a, thereby actuating the valve to release medicament through its stem block nozzle 415a. Medicament release from each container 420a will interfere with the beam of infrared radiation from its respective emitter (not shown) resulting in a reduction in radiation reaching its detector 450a.

Control circuitry is also provided which includes power supply 460 (e.g. a voltaic cell or battery of voltaic cells) with regulator and filter 462 and a switch 465 in the form of a solid state switching device. The switch 465 itself connects to control circuitry including micro-controller 470 which has an analogue and digital interface and connects with pressure transducer 471 which has an input in the form of a pressure tube 472 located within the dispensing outlet 416 of the inhaler housing 410.

On actuation, the patient inhales through the outlet 416 resulting in a change in pressure within the housing 410 and pressure tube 472. The change in pressure is detected by the pressure transducer 471 which sends a signal to the micro-controller 470. Release of each medicament is sensed when interference of the infra red beam emitted by the emitter (not shown) is detected by each detector 450a and a signal sent to the micro-controller 470 which can be configured to carry out one or more tasks. For example it may be configured to display an error message if the medicament is not dispensed.

The micro-controller 470 is connected to a display 474 for display of information to the patient and also with a computer interface 475 for exchange of data therewith. Communication with the computer interface 475 may be via a wired, optical or radio communications link. The micro-controller 470 is also connected to shake detector 476 for use in detecting whether the containers 420a are shaken prior to actuation of the dispenser and to a clock-calendar module 477 including a temperature sensor. All circuitry and components thereof including the power supply 460, display 474, shake detector 476, computer interface 475 and clock-calendar module 476 may be arranged to be present on the housing 410 such that the system is in the form of a discrete, hand-held device.

Whilst the system of FIG. 5 has been described in detail in respect of a metered dose inhaler it will be appreciated that identical sensors could be attached to a dry powder inhaler (DPI) device in a similar fashion.

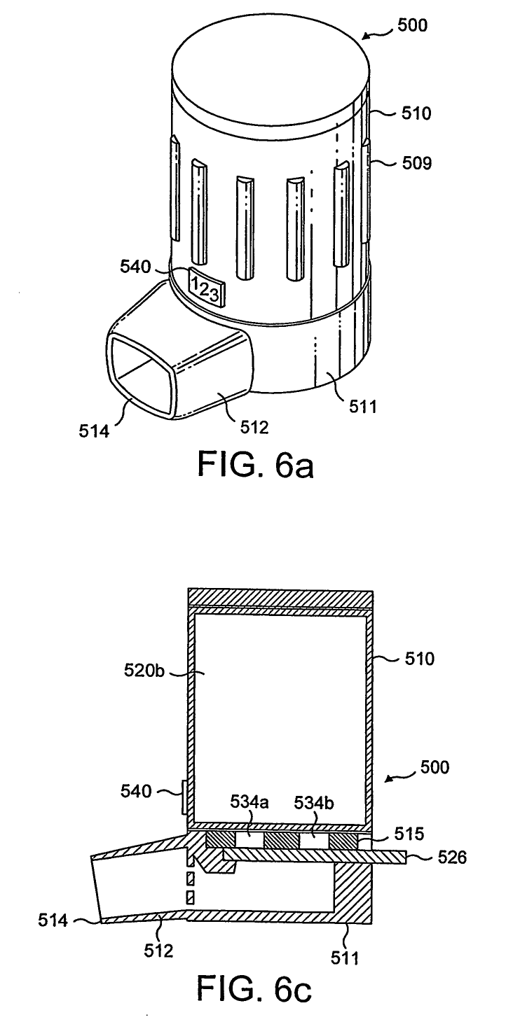

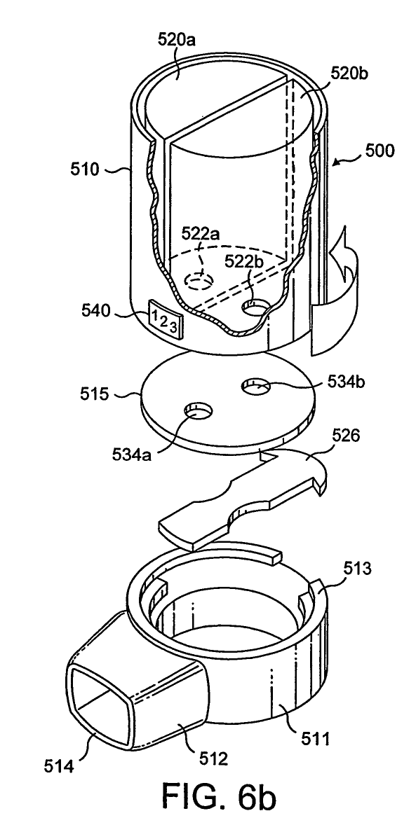

FIGS. 6a to 6c illustrate a reservoir dry powder inhaler (RDPI) herein, as shown respectively in perspective, exploded and sectional side views. The dispenser comprises a generally L-shaped body 500 comprised of upper column-shaped housing 510 rotationally mounted to base 511. The base 511 is shaped to define a common outlet 512 in the form of a mouthpiece 514. The column-shaped housing 510 has grips 509 for ease of patient grip, and is provided with two medicament containers 520a, 520b (both visible in FIG. 6b only) of semi-circular cross-section, each for containment of dry powder medicament. The first medicament container 520a contains first active medicament component. The second medicament container 520b contains second active medicament component. Each container 520a, 520b is itself provided with circular delivery orifice 522a, 522b for delivery of its dry powder medicament contents. Locating within the upper rim 513 of the base 511 and fixedly mounted with respect thereto, there is provided circular plate 515. The plate has two circular metering orifices 534a, 534b, each sized and shaped to register with the circular delivery orifices 522a, 522b of the respective containers 520a, 520b, in a metering position. Dispensing lever 526 locates beneath the plate 515 and is mounted for rotation with respect to the. base 511. The Lever is rotationally movable from a non-dispensing position in which it acts to close off communication between the metering orifices 534a, 534b of the plate 515 to a dispensing position in which the metering orifices 534a, 534b communicate with the common outlet 512 and mouthpiece 514 of the base 511 for dispensing of medicament therethrough. Actuation indicator 540 is located on column 510 and arranged to be responsive to rotatory movement thereof.

Usage of the dispenser of FIGS. 6a to 6c involves two distinct actions, namely metering and dispensing. In the metering action, the column 510 is rotated with respect to the base 511 until the circular delivery orifices 522a, 522b of the respective containers 520a, 520b are brought into registration with the circular metering orifices 534a, 534b of the plate 515. A metered quantity of the medicament powder contents of each container 520a, 520b is thereby delivered under gravity to each metering orifice 534a, 534b. This rotatory action of the column 510 also results in advancement of the actuation indicator 540 by one dose count. The count therefore relates to the metering of medicament from both medicament containers 520a, 520b. The column 511 is then rotated in a reverse sense to bring the respective orifices 522a, 522b and 534a, 534b out of registration with each other but leaving a metered quantity of medicament powder in each metering orifice 534a, 534b. It will be appreciated that in the metering stage, the lever 526 is in the non-dispensing (i.e. closed off) position with respect to the plate 515.

In the dispensing action, the lever 526 is now rotated from the non-dispensing position in which it acts to close off communication between the metering orifices 534a, 534b of the plate 515 to the dispensing position in which the volume of medicament powder contained within each metering orifice 534a, 534b is released to the base for dispensing to an inhaling patient through the common outlet 512 and mouthpiece 514.

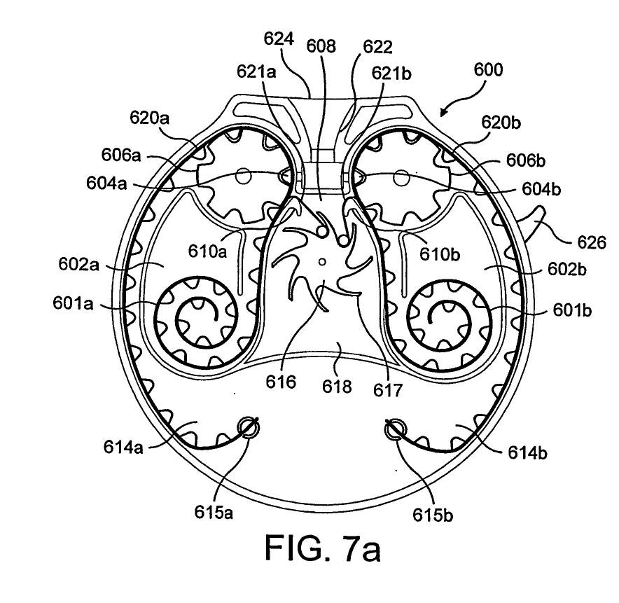

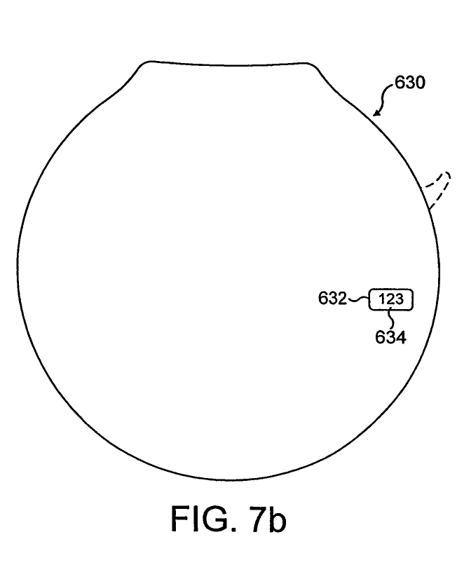

FIG. 7a illustrates a sectional view of base unit 600 of a medicament dispenser according to the invention. FIG. 7b illustrates a protective cover unit 630 provided for mounting to the base unit 600.

In respect of the base unit 600, first and second medicament-containing blister strips 601a, 601b are positioned within respective left and right chambers 602a, 602b of the base unit 600. Each blister strip 601a, 601b engages in respective multi-pocket index wheel 606a, 606b, and successive pockets are thereby guided towards a central opening station 608. The rotation of the index wheels 606a, 606b is optionally coupled together. At the opening station 608, the lid foil 620a, 620b and base foil 621a, 621b parts of each strip 601 a, 601b are peelably separable about beak 610a, 610b. The resulting empty base foil 621a, 621b coils up in respective base take-up chambers 614a, 614b. A base foil anchor 615a, 615b anchors the end of each respective base foil 621a, 621b in its chamber 614a, 614b. The used lid foil 620a, 620b feeds over its respective beak 610a, 610b and coils about common lid take-up spindle 616 in the common lid take-up chamber 618.

It will be noted that common lid take-up spindle 616 comprises plural arms 617 that splay out radially from the centre to give it an overall ‘collapsible wheel’ form. In use, as lid-foil 620a, 620b wraps around the spindle 616, the arms 617 collapse inwardly thereby reducing the diameter of the spindle 616 itself but acting to maintain a roughly constant effective winding diameter as defined by the diameter of the spindle 616 in combination with the used lid foil 620a, 620b wrapped there around. The maintenance of this constant effective winding diameter ensures uniform indexing of each strip 601a, 601b over the entire strip length.

The cover unit 630 is shaped for mating engagement with the base unit such that an overall ‘clam-shell’ casing is defined. The cover unit 630 is provided with a viewing window 632 through which count indicia 634 are visible. The count indicia 634 comprise the display of an actuation indicator (workings not visible) provided to the dispenser and responsive to actuation thereof, as described below.

In use, the dispenser is primed by actuating lever 626 located on the side of the dispenser to drivably actuate the lid-take up spindle 616 to advance each blister strip 601a, 601b, thereby causing the leading pocket 604a, 604b thereof to be peeled open. Movement of the actuating lever 626 also acts such as to drive on the actuation indicator (not visible) such that the count indicia 634 increase by one unit. It may be appreciated that the count relates to the advancement and leading pocket 604a, 604b opening of both strips 601a, 601b since the movement of both is responsive to the movement of the single lever 626 and single lid-take up spindle 616.

To inspire the contents of the opened pockets 604a, 604b, the patient then breathes in through the outlet 624. This results in negative pressure being transmitted through manifold 622 to the opened leading pocket 604a, 604b of each strip 601a, 601b at the opening station 608. This in turn, results in the medicament powder contained within each of the opened pockets 604a, 604b being drawn out through the common manifold 622 to the outlet 624 and hence to the patient as an inhaled combination medicament dose. It be appreciated that, mixing of each separately delivered component of the combined medicament product happens as the powder is transported from each opened pocket 604a, 604b to the outlet 624.

Importantly, the dispenser of FIG. 9 enables different medicament types to be stored separately in each of the strips 601a, 601b but allows for the release and delivery thereof to the patient via the single outlet 624 as a combined inhaled product.

It may be appreciated that any of the parts of the device or any medicament thereof which contacts medicament may be coated with materials such as fluoropolymer materials (e.g. PTFE or FEP) which reduce the tendency of medicament to adhere thereto. Any movable parts may also have coatings applied thereto which enhance their desired movement characteristics. Frictional coatings may therefore be applied to enhance frictional contact and lubricants (e.g. silicone oil) used to reduce frictional contact as necessary.

The device of the invention is suitable for dispensing medicament combinations, particularly for the treatment of respiratory disorders such as asthma and chronic obstructive pulmonary disease (COPD), bronchitis and chest infections. Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α-propionyloxy-androsta-1,4-diene-17 β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Preferred components of the combinations comprise medicaments selected from albuterol, salmeterol, fluticasone propionate and beclomethasone dipropionate and salts or solvates thereof, e.g., the sulphate of albuterol and the xinafoate of salmeterol.

Preferred components of combinations of active ingredients contain a bronchodilator in combination with an anti-inflammatory. The bronchodilator is suitably a beta-agonist, particularly a long-acting beta-agonist (LABA). Suitable bronchodilators include salbutamol (e.g., as the free base or the sulphate salt), salmeterol (e.g., as the xinafoate salt) and formoterol (eg as the fumarate salt). The anti-inflammatory is suitably an anti-inflammatory steroid. Suitably anti-inflammatory compounds include a beclomethasone ester (e.g., the dipropionate), a fluticasone ester (e.g., the propionate) or budesonide or any salt or solvate thereof. One preferred combination of components comprises fluticasone propionate and salmeterol, or any salt or solvate thereof (particularly the xinafoate salt). A further combination of components of particular interest is budesonide and formoterol or any salt or solvate thereof (e.g. formoterol as the fumarate salt).

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with excipients (carriers) which are suitable for inhalation. Suitable excipients include organic excipients such as polysaccharides (i.e. starch, cellulose and the like), lactose, glucose, mannitol, amino acids, and mattodextrins, and inorganic excipients such as calcium carbonate or sodium chloride. Lactose is a preferred excipient.

Particles of powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The excipient may be included with the medicament via well-known methods, such as by admixing, co-precipitating and the like. Blends of excipients and drugs are typically formulated to allow the precise metering and dispersion of the blend into doses. A standard blend, for example, contains 13000 micrograms lactose mixed with 50 micrograms drug, yielding an excipient to drug ratio of 260:1. Dosage blends with excipient to drug ratios of from 100:1 to 1:1 may be used. At very low ratios of excipient to drug, however, the drug dose reproducibility may become more variable.

Aerosol formulations suitable for use with metered dose inhaler (MDI) dispensers typically comprise a propellant. Suitable propellants include P11, P114 and P12, and the CFC-free hydrofluoroalkane propellants HFA-134a and HFA-227.

The MDI aerosol formulation may additionally contain a volatile adjuvant such as a saturated hydrocarbon for example propane, n-butane, isobutane, pentane and isopentane or a dialkyl ether for example dimethyl ether. In general, up to 50% w/w of the propellant may comprise a volatile hydrocarbon, for example 1 to 30% w/w. However, formulations, which are free or substantially free of volatile adjuvants are preferred. In certain cases, it may be desirable to include appropriate amounts of water, which can be advantageous in modifying the dielectric properties of the propellant.

A polar co-solvent such as C₂₋₆ aliphatic alcohols and polyols e.g. ethanol, isopropanol and propylene glycol, preferably ethanol, may be included in the MDI aerosol formulation in the desired amount to improve the dispersion of the formulation, either as the only excipient or in addition to other excipients such as surfactants. Suitably, the drug formulation may contain 0.01 to 30% w/w based on the propellant of a polar co-solvent e.g. ethanol, preferably 0.1 to 20% w/w e.g. about 0.1 to 15% w/w. In aspects herein, the solvent is added in sufficient quantities to solubilise the part or all of the medicament component, such formulations being commonly referred to as solution formulations.

A surfactant may also be employed in the MDI aerosol formulation. Examples of conventional surfactants are disclosed in EP-A-372,777. The amount of surfactant employed is desirable in the range 0.0001% to 50% weight to weight ratio relative to the medicament, in particular, 0.05 to 5% weight to weight ratio.

The final aerosol formulation desirably contains 0.005-10% w/w, preferably 0.005 to 5% w/w, especially 0.01 to 1.0% w/w, of medicament relative to the total weight of the formulation.

The device of the invention is in one aspect suitable for dispensing medicament for the treatment of respiratory disorders such as disorders of the lungs and bronchial tracts including asthma and chronic obstructive pulmonary disorder (COPD). In another aspect, the invention is suitable for dispensing medicament for the treatment of a condition requiring treatment by the systemic circulation of medicament, for example migraine, diabetes, pain relief e.g. inhaled morphine.

Accordingly, there is provided the use of a device according to the invention for the treatment of a respiratory disorder, such as asthma and COPD. Alternatively, the present invention provides a method of treating a respiratory disorder such as, for example, asthma and COPD, which comprises administration by inhalation of an effective amount of medicament product as herein described from a device of the present invention.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. A medicament dispenser device for use in the delivery of a combination medicament product, the device comprising:
a first medicament container for containing a first medicament component;
a first release means for releasing the contents of said first medicament container;
at least one further medicament container for containing at least one further medicament component; and
at least one further release means for releasing the contents of each said at least one further medicament container;
wherein the first medicament component is kept separate from the at least one further medicament component until the point of release thereof for delivery in combination, and wherein the dispenser device additionally comprises:
at least one actuation indicator associated with the first medicament container and/or the at least one further medicament container.

2. A medicament dispenser device according to claim 1, wherein the device comprises only at least one further medicament dispenser.

3. A medicament dispenser device according to claim 1, wherein the at least one actuation indicator associates directly with the first medicament container and the at least one further medicament container.

4. A medicament dispenser device according to claim 1, wherein the at least one actuation indicator associates indirectly with the first medicament container and the at least one further medicament container.

5. A medicament dispenser device according to any of claim 1, wherein a single actuation indicator associates with both the first medicament container and the at least one further medicament container.

6. A medicament dispenser device according to claim 1, wherein a single actuation indicator associates with the first medicament container only and the first medicament container and at least one further medicament container are coupled such that actuation of the first medicament container also results in actuation of the at least one further medicament container.

7. A medicament dispenser device according to claim 6, wherein the dispenser device comprises an actuator coupling that acts such as to couple an actuating movement of the first and the at least one further medicament container.

8. A medicament dispenser device according to claim 7, wherein said coupling actuator comprises a lever arranged to act on both the first and the at least one further medicament container to enable release of medicament therefrom.

9. A medicament dispenser device according to claim 6, wherein the dispenser device comprises fixing means for fixedly coupling the movement of the first and the at least one further medicament container together.

10. A medicament dispenser device according to claim 6, wherein the dispenser device comprises a meter coupling that acts such as to couple a metering movement relative to the first and the at least one further medicament container.

11. A medicament dispenser device according to claim 10, wherein the first and the at least one further medicament container each have bulk reservoir form and said metering movement is that of one or more metering means moveable relative to the first and the at least one further medicament container for metering medicament therefrom.

12. A medicament dispenser device according to claim 6, wherein the dispenser device comprises a dose advancement coupling that acts such as to couple a dose advancement movement relative to the first and the at least one further medicament container.

13. A medicament dispenser device according to claim 12, wherein the first and the at least one further medicament container each comprise a pack carrying multiple individual doses of medicament and said dose advancement movement results in advancement of a next accessible dose of each pack.

14. A medicament dispenser device according to claim 6, wherein the dispenser device comprises a dose access coupling that acts such as to couple a dose access movement relative to the first and the at least one further medicament container.

15. A medicament dispenser device according to claim 14, wherein the first and the at least one further medicament container each comprise a pack carrying multiple individually accessible doses of medicament and said dose access movement enables access to a next accessible dose of each pack.

16. A medicament dispenser device according to claim 6, wherein the single actuation indicator is arranged to detect an actuation step relating to the first medicament container.

17. A medicament dispenser device according to claim 6, wherein the single actuation indicator is arranged to detect the release of medicament from one medicament container.

18. A medicament dispenser device according to claim 10, wherein the single actuation indicator is arranged to detect a pre-actuation step relating to the first medicament container.

19. A medicament dispenser device according to claim 1, wherein the first and the at least one further medicament container each have an actuation indicator associated therewith.

20. A medicament dispenser device according to claim 19, wherein each actuation indicator is arranged to detect an actuation step relating to each medicament container.

21. A medicament dispenser device according to claim 19, wherein each actuation indicator is arranged to detect a pre-actuation step relating to each medicament container.

22. A medicament dispenser device according to claim 19, wherein each actuation indicator is arranged to detect medicament release from each medicament container.

23. A medicament dispenser device according to claim 1, wherein the at least one actuation indicator comprises an actuation sensor.

24. A medicament dispenser device according to claim 23, wherein the actuation sensor senses a parameter selected from the group consisting of electro magnetic radiation, magnetic field, light, motion, temperature, pressure, sound, oxygen concentration, carbon dioxide concentration, moisture and any combinations thereof.

25. A medicament dispenser device according to claim 1, wherein the at least one actuation indicator comprises a release sensor for detecting medicament release from the first and/or at least one further medicament container.

26. A medicament dispenser device according to claim 1, wherein the actuation indicator comprises a visual display unit for display of dose count information.

27. A medicament dispenser device according to claim 1, wherein the device is in the form of an inhaler device.

28. A medicament dispenser according to claim 27, wherein the inhaler device is in the form of a device selected from the group consisting of a reservoir dry powder inhaler (RDPI), a multi-dose dry powder inhaler (MDPI), a metered dose inhaler (MDI), a liquid spray inhaler (LSI) and any hybrids thereof.

29. A medicament dispenser according to claim 27 comprising a single outlet for delivery of the combination medicament product to the respiratory tract of a patient.

30. A medicament dispenser according to claim 27, actuable in response to an inward breath of a patient.

31. A medicament dispenser according to claim 1, wherein the first medicament container contains a first medicament component and the at least one further medicament container contains at least one further medicament component.

32. A medicament dispenser according to claim 31, wherein said first medicament component comprises a bronchodilator and said at least one further medicament component comprises an anti-inflammatory.

33. A medicament dispenser according to claim 32, wherein said bronchodilator is a beta-agonist and said anti-inflammatory is a steroid.

34. A medicament dispenser according to claim 33, wherein said bronchodilator is selected from the group consisting of salbutamol, salmeterol, formoterol and any salts or solvates thereof and mixtures thereof.

35. A medicament dispenser according to claim 33, wherein said anti-inflammatory is selected from the group consisting of a beclomethasone ester, fluticasone ester, budesonide and any salt or solvates thereof and mixtures thereof.

36. (canceled)

37. A method of treating a respiratory disorder in a patient in need thereof comprising administering an effective amount of medicament suitable for treating said respiratory disorder to the patient by inhalation from a medicament dispenser according to claim 1.
